(19) United States
(12) Patent Application Publication
(10) Pub. No.: US 2012/0214167 A1
(43) Pub. Date: Aug. 23, 2012

(54) APPARATUS FOR BIO-AUTOMATION

(75) Inventors: David Thomson, Hamilton Queensland (AU); Jonathan Salmon, Makati City (PH)

(73) Assignee: ITI SCOTLAND LIMITED, Glasgow, GB (GB)

(21) Appl. No.: 13/387,525

(22) PCT Filed: Jul. 27, 2010
(86) PCT No.: PCT/EP10/60885

§ 371 Date: May 14, 2012

(30) Foreign Application Priority Data
Jul. 29, 2009 (GB) 0913229.1

(51) Int. Cl. C12Q 1/68; C12Q 1/52; G01N 33/53; B01L 99/00; C12M 1/40

(52) U.S. Cl. 435/6.12; 422/544; 435/289.1; 422/69; 435/287.1; 435/287.2; 435/16

(57) ABSTRACT

Provided is a processing apparatus for enabling a plurality of processes to be conducted on a sample within a microfluidic or nanofluidic cartridge, the apparatus comprising:
a) a plurality of processing modules for facilitating the processes conducted on the sample within the cartridge;
b) a coupling means for reversibly coupling the cartridge to the apparatus;
c) a transport means capable of bringing the cartridge coupled to the coupling means into communication with each of the processing modules;
   wherein the plurality of processing modules includes a component isolation module capable of facilitating a separation process within the cartridge, a manipulator module capable of facilitating the movement of fluids within the cartridge; and an optical module capable of interacting optically with the cartridge.

### FIELD OF THE INVENTION

The present invention concerns a processing apparatus for conducting assays on biological samples in the medical environment. The apparatus is adapted to interact with and, in particular, control analytical processes conducted on samples within disposable microfluidic devices, such as disposable cartridges.

### BACKGROUND OF THE INVENTION

Conventional medical assays require one or more samples (such as blood or urine samples) to be taken from a patient in a hospital, or in a doctor's surgery, and then transferred to a laboratory for analysis. In the past, analysis of a sample in a “central” laboratory was unavoidable, due to the size and complexity of assay devices and systems. However, the requirement to analyse the sample in a remote location causes significant delay in diagnosing and treating a patient. In order to reduce the delay, there is an ongoing need to develop assay systems and methods that can be carried out in the near-patient environment (at the point of care) and that provide results quickly. Over time, smaller and less costly assay devices have been developed for this purpose.

It has been known for some time to employ cartridge arrangements in biological assay systems. Cartridges are advantageous in that they allow use of a single generalised assay device to assay for a number of different analytes by employing a different cartridge for each different analyte. They also simplify the assay procedure, in comparison with larger, more cumbersome laboratory systems. The development of microfluidic processing devices and chips has facilitated the development of such cartridges, since microfluidics allows much smaller (and cheaper) cartridges to be produced which can readily be inserted into a larger robust assay device.

Published application US 2004/0086872 describes a system for microfluidic processing and/or analysis of nucleic acid in a sample. In particular, the system comprises a cartridge configured to receive the sample, and a control apparatus that interfaces electrically, and, optionally mechanically, optically, and/or acoustically with the cartridge. The control apparatus includes a controller that processes digital information and sends and receives electrical signals to co-ordinate electrical, mechanical and/or optical activities performed by the control apparatus and the cartridge.

However such systems are relatively limited in the range of assays/analytical tests that they can perform and therefore they do not fully meet the needs of an assay system for the near-patient environment. It is the aim of the present invention to address this issue. Specifically, it is an aim of the present invention to provide an improved processing apparatus which is capable of conducting a range of assays on biological samples.

### SUMMARY OF THE INVENTION

Accordingly, the present invention provides a processing apparatus for enabling a plurality of processes to be conducted on a sample within a microfluidic or nanofluidic cartridge, the apparatus comprising:
a) a plurality of processing modules for facilitating the processes conducted on the sample within the cartridge;
b) a coupling means for reversibly coupling the cartridge to the apparatus;
c) a transport means capable of bringing the cartridge coupled to the coupling means into communication with each of the processing modules;
   wherein the plurality of processing modules includes a component isolation module capable of facilitating a separation process within the cartridge, a manipulator module capable of facilitating the movement of fluids within the cartridge; and an optical module capable of interacting optically with the cartridge.

In a preferred aspect of the invention the plurality of processing modules further includes a PCR module capable of facilitating a PCR within the cartridge.

In a further preferred aspect of the present invention the processing apparatus further comprises a microfluidic control module, which is coupled to the cartridge when the cartridge is coupled to the apparatus, and which is capable of facilitating the flow of fluid within the cartridge via the use of pressure and vacuum sources.

The present inventors have found that a processing apparatus arranged as described above can be used in combination with disposable microfluidic or nanofluidic cartridges to conduct a variety of analytical tests on a sample within the cartridge. The processing apparatus is particularly advantageous in its flexibility, allowing different analytical tests to be performed on the same platform. For example, a cartridge containing the necessary components to assay for a cancer cell marker can be run on the processing apparatus. Once this assay is completed, and the used cartridge removed, a further cartridge containing the necessary components to assay for an infectious disease can be inserted and run on the same processing apparatus.

The present invention further provides an assay method for detecting and optionally quantifying one or more analytes in a sample, which method comprises:
(a) introducing the sample into a microfluidic or nanofluidic cartridge;
(b) coupling the cartridge to the coupling means of a processing apparatus as described herein; and
(c) assaying for one or more analytes by bringing the cartridge into communication with at least two of the processing modules of the processing apparatus.

Still further the present invention provides the use of a processing apparatus as described herein for detecting and optionally quantifying one or more analytes in a sample present in a microfluidic or nanofluidic cartridge.

### DETAILED DESCRIPTION OF THE INVENTION

The present invention will now be described further by way of example only with reference to the accompanying figures, in which:

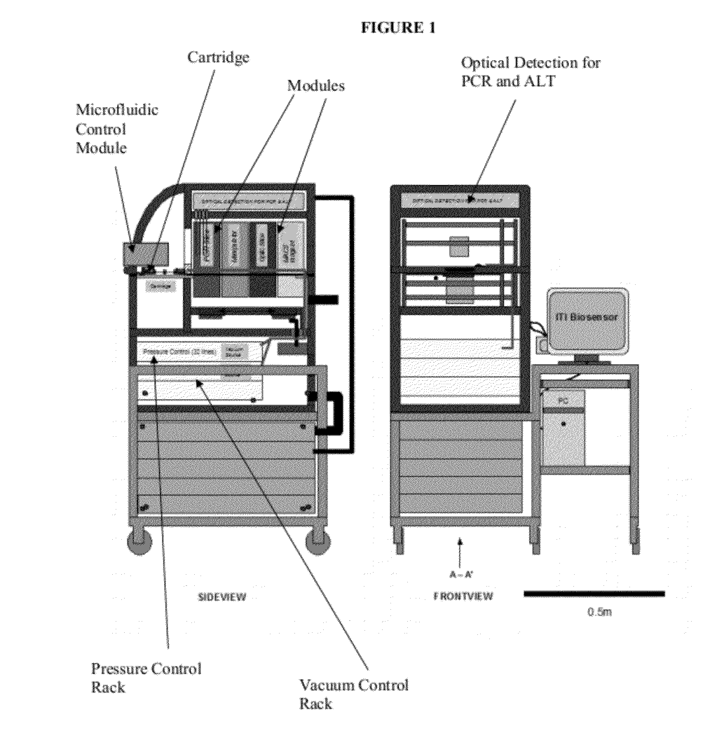

FIG. 1 illustrates an example of the processing apparatus of the present invention attached to a disposable microfluidic cartridge.

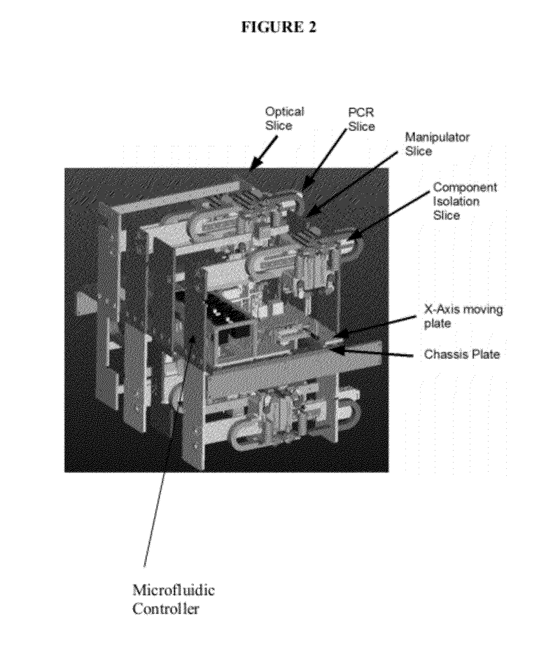

FIG. 2 shows the design of the processing apparatus in a possible embodiment of the present invention. The Figure illustrates the position of the modules within the apparatus as well as a moving plate which acts as the transport means.

As indicated above, the present invention relates to a processing apparatus comprising a plurality of processing modules or slices. These modules are capable of facilitating and/or controlling assay methods conducted within microfluidic or nanofluidic cartridges which can be attached to the apparatus. In particular the processing modules are hardware slices which can control general functions, assay method steps, or parts of assay method steps which are applicable to a wide variety of assay formats. Examples of such functionalities are valve actuation, application of a pressure or a vacuum, temperature increase, decrease or stabilisation, application of a moving or stationery magnetic force, illumination with light at a variety of wavelengths, application of a static or dynamic mechanical force, and detection of emitted, reflected or transmitted light.

The ability to facilitate and/or control the above listed functions is divided between the modules. However, together the modules have the ability to facilitate and/or control an entire assay method including the sample preparation, the assay method steps, and the detection of the end result of the assay, e.g. the presence or absence of an analyte.

In particular, the modules of the apparatus include a component isolation module capable of facilitating a separation process within the cartridge, a manipulator module capable of facilitating the movement of fluids within the cartridge, an optical module capable of interacting optically with the cartridge, and, optionally, a thermo cycling module capable of facilitating and/or running a qPCR cycle on the cartridge.

All or some of the above modules may be capable themselves of facilitating fluid flow in the microfluidic cartridge via the application of pressure or vacuum.

Alternatively, as is preferred, the apparatus comprises a microfluidic control module. It is particularly preferred that this module is coupled to the cartridge at all times while the cartridge is coupled to the apparatus. In this embodiment the microfluidic control module is a module which is separate and/or distinct from the other processing modules. Alternatively, there may be one or more microfluidic control modules which are integrated with the other processing modules. In this arrangement the microfluidic control module(s) is either permanently or reversibly coupled to the other processing modules.

The component isolation module can be used to facilitate and/or control the initial preparation of the sample within the cartridge. In particular, this preparation is frequently the separation off of a particular part of the sample to be used in the assay method. In one embodiment the separation step includes the use of magnetic beads contained within the cartridge and therefore the module is capable of controlling the application of a magnetic force to an area of the cartridge so that the beads can be moved within the cartridge chambers or fluid channels.

The manipulator module can be used to facilitate the assay method steps. This module may also be capable of controlling the application of a magnetic force to an area of the cartridge to move magnetic beads. The manipulator module may additionally or alternatively be also capable of controlling the application of a mechanical force. This force could be in order to actuate a valve, move a fluid or control the contact of a vibratory mixing device to the cartridge.

The optical module comprises an illumination means and is capable of subjecting the cartridge to particular optical conditions, e.g. illuminating a reaction chamber within the microfluidic cartridge with light of a particular wavelength, and then capturing an image of the chamber with an image capture means or transducing an optical signal. This enables fluorescence or colour changes within the reaction chamber to be detected. Alternatively a point detection method may be used to perform this transduction. That transduction could be any number of optical detection methods known in the art such as Fluorescence, Raman spectroscopy, TIRF detection or FLIM. The optical transduction is preferably non contact; however this is not a requirement of the module. In the preferred embodiment the detection is an inverted epifluorescent microscope design with a filtered CCD camera and a more sensitive point detector module in parallel.

In a preferred aspect of the present invention the apparatus further comprises a PCR module capable of facilitating a PCR within the cartridge. This module is in particular capable of controlling the temperature within a reaction chamber in the cartridge so as to deliver the thermal cycling required for a PCR. In a preferred embodiment this module is capable of quantitative PCR by means of a built in fibre optic detection system.

As indicated above, in a further preferred aspect of the invention the processing apparatus further comprises a microfluidic control module, which is capable of facilitating the movement of fluid in the cartridge. The microfluidic control is preferred by the inventors to be independent of the modules used to manipulate or probe the microfluidic chip. This allows all modules simultaneous access to the microfluidic control. In a preferred embodiment of this aspect of the invention the component isolation, manipulator, optical and PCR modules do not interact with the cartridge electrically and do not have the ability to control the microfluidic pressure and vacuum. Instead the microfluidic control module has this control.

The microfluidic module is preferably located as close to the microfluidic device as possible. In one embodiment the microfluidic module can be part of the transport means.

The microfluidic control module is preferably coupled to the cartridge when the cartridge is reversibly coupled to the processing apparatus.

The specificities of an individual assay, such as the components or reagents used, are provided by the microfluidic or nanofluidic cartridge, which is not the subject of the present invention. Microfluidic and nanofluidic devices are well known in the art, and are designed to manipulate fluids (liquids and/or gases) that are constrained in the microscale or nanoscale respectively. Microfluidic and nanofluidic devices have been used in many different fields which require the use of very small volumes of fluids, including engineering and biotechnology. Suitable microfluidic and nanofluidic cartridges which can be used in the present invention are known in the art. In particular, it is preferred that the cartridge used is one as described in WO 2008/037995.

The cartridges are reversibly attached to the processing apparatus of the invention via a coupling means. The coupling means is not particularly limited except that it must be capable of reversibly attaching the cartridge to the apparatus, so that the cartridge can be removed once the assay has been completed. Suitable snap fittings are known in the art. The coupling means itself is attached to a transport means which is capable of moving the coupling means with the cartridge attached between the different modules of the apparatus. In one embodiment the transport means is a moving plate, as illustrated in FIG. 2.

In particular, the transport means is capable of bringing the cartridge into communication with each of the processing modules, so that the module can control a particular aspect of the assay method being conducted. In one aspect of the invention the modules are aligned within in the processing apparatus so that the cartridge can only be brought into communication with one module at a time. In another aspect of the invention the modules are arranged so that the cartridge can be brought into communication with at least two modules at a time. Preferably one of these two modules is the optical module.

In a further aspect of the invention the processing apparatus comprises a control apparatus which controls the interaction of the modules with the cartridge, and includes a user interface. The user interface allows the user to control the assay performed by the apparatus, and to input information which may alter particular aspects of the assay. This allows for further flexibility in the nature of the assay performed by the processing apparatus. The results of the assay may also be presented via the user interface.

The present invention further provides an assay method for detecting and optionally quantifying one or more analytes in a sample, which method comprises:
(a) introducing the sample into a microfluidic or nanofluidic cartridge;
(b) coupling the cartridge to the coupling means of a processing apparatus as described herein; and
(c) assaying for one or more analytes by bringing the cartridge into communication with at least two of the processing modules of the processing apparatus.

The types of assays which may be performed using the processing apparatus are not particularly limited. Accordingly, the assays may be for screening, purifying, identifying, capturing and/or quantifying any type of substance and in particular any type of biological substance which may be present in the sample.

The type of biological substance may be a pathogen that causes infection (such as a virus, a bacterium, a fungal agent or the like) or may be a biological characteristic of the patient (such as gene profiling, protein profiling, disease and prognosis profiling or the like—these may include DNA, RNA, protein, polypeptide, peptide, and enzyme assays, for example) or may even be a biologically significant chemical (such as small molecules, metabolites, pharmaceuticals and drugs). The assays are typically employed to test a patient in order to establish a diagnosis.

Particularly preferred assays which can be performed using the processing apparatus are:
1. Nucleic acid assays (such as DNA or RNA).
2. Enzyme assays (an ALT (Alanine Aminotransferase, a liver enzyme) assay is especially preferred in the context of hepatitis infection).
3. Protein assays (typically using antibodies for detection, e.g. on a microarray—preferred analytes of interest include hepatitis (A, B and/or C) and interferon gamma (IFN-β).
4. Small molecule assays (such as pharmaceuticals or drugs—typical methods involve competition assays using antibodies). Therapeutic drug monitoring (TDM) is also an option.

### EXAMPLES

### Example 1

### Detection of the Liver Enzyme Alanine Aminotransferase in Human Blood

Sample: (spiked) Human Blood

Inputs: Sample prep Reagent, Reagent 1 and Reagent 2

Basic process:
1. Separate sample to get plasma
2. Mix R1, R2 and the plasma.
3. Observe a rate limited fluorescence output.

Modules: Microfluidic control module, Fibre coupled optics in the optical module, Controller, Component isolation module

Spiked human blood is injected into a microfluidic cartridge and the cartridge is attached to the processing apparatus. The processing apparatus brings the cartridge into communication with the component isolation module which, together with the microfluidic control module, controls the separation of the plasma from the blood sample.

The processing apparatus then brings the cartridge into communication with the optical module. The regents, which are already present within the cartridge, are mixed with the plasma and the rate limited fluorescence output is detected by the fibre coupled optics within the optical module.

### Example 2

### Detection of TPO

Sample: Blood (spiked)

Inputs: Sample Wash Buffer (SWB), Conjugate Wash Buffer (CWB), Conjugate, MUP, PEG and Rabbit-IgG, Sample Prep Reagents

Basic process:
1. Separate sample to get plasma
2. Fill cartridge compartment with SWB
3. Introduce beads
4. Trap beads in channels by closing valves. Bring magnet to capture.
5. Add sample and/or control to each correspondent channel. Release beads, incubate.
6. Wash with SWB
7. Add conjugate and incubate
8. Wash with CWB
9. Add MUP monitor fluorescence.

Spiked blood is injected into a microfluidic cartridge and the cartridge is attached to the processing apparatus. The processing apparatus brings the cartridge into communication with the component isolation module which, together with the microfluidic control module, controls the separation of the plasma from the blood sample.

The processing apparatus then brings the cartridge into communication with the manipulator module which, together with the microfluidic control module, control steps 2 to 8 of the process.

The processing apparatus then brings the cartridge into communication with the optical module. The microfluidic control module controls the addition of MUP to the sample and the optical module monitors the fluorescent output.

### Example 3

### Detection of RNA

Sample: Blood (spiked with MLV-E (or Hepatitis C Virus (HCV)) pseudoparticles)

Inputs: MagMAX beads+lysis buffer, MagMAX wash buffer 2, MagMAX elution buffer, RT mix, Chargeswitch beads+Binding buffer, Chargeswitch wash buffer, PCR mix (mix1, mix2, +ve control, −ve control).

Basic process:
1. Separate sample to get plasma
2. Introduce Beads+lysis buffer. Introduce Plasma. MIX
3. Transfer to 2^{nd} device and capture beads
4. Wash beads
5. Mix beads with elution buffer (release beads). Recapture beads
6. Introduce RT reagents, and MIX with eluted RNA
7. Introduce CS-beads+binding buffer
8. MIX and transfer to PCR chambers
9. Elute cDNA in PCR buffer
10. qPCR

Spiked blood is injected into a microfluidic cartridge and the cartridge is attached to the processing apparatus. The processing apparatus brings the cartridge into communication with the component isolation module which, together with the microfluidic control module, controls the separation of the plasma from the blood sample.

The processing apparatus then brings the cartridge into communication with the manipulator module which, together with the microfluidic control module, controls steps 2 to 8 of the process.

The processing apparatus then brings the cartridge into communication with the PCR module which, together with the microfluidic control module, controls steps 9 and 10 and performs PCR on the sample. The qPCR processor monitors the fluorescence of the chamber. This fluorescence and its kinetics provide the result of the assay.

1. A processing apparatus for enabling a plurality of processes to be conducted on a sample within a microfluidic or nanofluidic cartridge, the apparatus comprising:
(a) a plurality of processing modules for facilitating the processes conducted on the sample within the cartridge;
(b) a coupling means for reversibly coupling the cartridge to the apparatus;
(c) a transport means capable of bringing the cartridge coupled to the coupling means into communication with each of the processing modules;
wherein the plurality of processing modules includes a component isolation module capable of facilitating a separation process within the cartridge, a manipulator module capable of facilitating the movement of fluids within the cartridge; and an optical module capable of interacting optically with the cartridge.

2. A processing apparatus according to claim 1 wherein the plurality of processing modules further includes a PCR module capable of facilitating a PCR within the cartridge.

3. A processing apparatus according to claim 1, claim 2 wherein the processing apparatus further comprises a microfluidic control module.

4. A processing apparatus according to claim 3 wherein the microfluidic control module is coupled to the cartridge when the cartridge is coupled to the coupling means.

5. A processing apparatus according to claim 1, wherein the modules are capable of interacting with the cartridge to provide valve actuation, to apply a pressure or a vacuum, to stabilise, increase or decrease temperature, to apply a static or dynamic magnetic force, to apply a static or dynamic mechanical force, to illuminate with light, or to detect emitted, reflected or transmitted light.

6. A processing apparatus according to claim 1, which further comprises a control apparatus comprising a user interface wherein the control apparatus is capable of sending and receiving electrical signals to/from the modules.

7. A processing apparatus according to claim 1, wherein the component isolation module is capable of facilitating the manipulation of magnetic beads within the cartridge.

8. A processing apparatus according to claim 1, wherein the manipulator module is capable of facilitating the manipulation of magnetic beads within the cartridge.

9. A processing apparatus according to claim 1, wherein the manipulator module is capable of facilitating an immunoassay or an enzymatic assay within the cartridge.

10. A processing apparatus according to claim 2, wherein the PCR module is capable of facilitating a quantitative PCR within the cartridge.

11. A processing apparatus according to claim 1, wherein the optical module includes an illumination means for exposing a part of the cartridge to light.

12. A processing apparatus according to claim 1, wherein the optical module includes an image capture means for capturing an image of a part of the cartridge.

13. A processing apparatus according to claim 1, wherein the cartridge to be coupled to the apparatus comprises a plurality of valves and at least one of the modules is capable of facilitating the operation of the valves.

14. An assay method for detecting and optionally quantifying one or more analytes in a sample, which method comprises:
(a) introducing the sample into a microfluidic or nanofluidic cartridge;
(b) coupling the cartridge to the coupling means of a processing apparatus according to claim 1; and
(c) assaying for one or more analytes by bringing the cartridge into communication with at least two of the processing modules of the processing apparatus.

15. An assay method according to claim 14, wherein the processing apparatus comprises a user interface to provide valve actuation, to apply a pressure or a vacuum, to stabilise, increase or decrease temperature, to apply a static or dynamic magnetic force, to apply a static or dynamic mechanical force, to illuminate with light, or to detect emitted, reflected or transmitted light according to claim 5 and wherein the assay method further comprises a step of selecting via the user interface the at least two processing modules to which the cartridge is brought into communication with during step (c).

16. A method for detecting and optionally quantifying one or more analytes in a sample present in a microfluidic or nanofluidic cartridge comprising using the apparatus of claim 1.
